(19) United States
(12) Patent Application Publication
(10) Pub. No.: US 2019/0293658 A1
(43) Pub. Date: Sep. 26, 2019

(54) DETERMINATION METHOD AND SYSTEM THEREOF

(71) Applicant: ProtectLife International Biomedical Inc., Taoyuan City (TW)

(72) Inventors: Chi-Chen YANG, Taoyuan City (TW); Po-Szu GU, Taoyuan City (TW); Hsiao-Chung TSAI, Taoyuan City (TW)

(21) Appl. No.: 16/361,228

(22) Filed: Mar. 22, 2019

(30) Foreign Application Priority Data
Mar. 23, 2018 (TW) 107110131

(51) Int. Cl. G01N 33/68; G01N 33/70

(52) U.S. Cl. CPC G01N 33/6827; G01N 2333/76; G01N 33/70; G01N 33/6839

(57) ABSTRACT

The present invention relates to a determination method and system thereof. The method includes: providing or receiving a urine sample; distributing the urine sample into a first urine sample and a second urine sample; measuring a total protein concentration of the first urine sample; measuring a creatinine concentration and an albumin concentration of the second urine sample; and calculating a urine protein to creatinine ratio (UPCR) and a urine albumin to creatinine ratio (UACR), in which the UPCR is defined as a ratio of the total protein concentration to the creatinine concentration, and the UACR is defined as a ratio of the albumin concentration to the creatinine concentration.

### CROSS-REFERENCE TO RELATED APPLICATION

This application claims priority to Taiwan Application Serial Number 107110131, filed Mar. 23, 2018, which is herein incorporated by reference.

### BACKGROUND

### Field of Invention

The present disclosure relates to a determination method and a system thereof, more particularly relates to a method and a system for determining the composition of urine.

### Description of Related Art

In recent years, a global increase in kidney disease incidence is observed along with changes in dietary habits and lifestyles. Chronic Kidney Disease (CKD) refers to abnormalities in renal function over a prolonged period. Generally, a patient is diagnosed with CKD if they have abnormalities of kidney functions for more than three months. CKD is divided into five stages based on the level of kidney function.

In some cases, abnormalities of kidney functions may lead to abnormalities in the contents of protein or some other substances in the urine. Therefore, the contents of these substances can be used as markers of CKD. For example, stage II CKD patients may experience albuminuria, while stage III CKD patients may experience proteinuria. However, in the current diagnosis of CKD, it lacks an effective approach to simultaneously determine stage II and stage III kidney diseases. Hence there is an urgent call for a novel determination method and system for determination of stage II and stage III kidney diseases.

### SUMMARY

One aspect of the present disclosure is to provide a determination method. The method includes: providing or receiving a urine sample; distributing the urine sample into a first urine sample and a second urine sample; measuring a total protein concentration of the first urine sample; measuring a creatinine concentration and an albumin concentration of the second urine sample; and calculating a urine protein to creatinine ratio (UPCR) and a urine albumin to creatinine ratio (UACR), in which the UPCR is defined as the ratio of the total protein concentration to the creatinine concentration, and the UACR is defined as the ratio of the albumin concentration to the creatinine concentration.

According to some embodiments of the present disclosure, the step of distributing the urine sample into the first urine sample and the second urine sample comprises distributing about 33% to about 80% of the urine sample by volume as the first urine sample and distributing about 20% to about 67% of the urine sample by volume as the second urine sample.

According to some embodiments of the present disclosure, the step of measuring the total protein concentration of the first urine sample comprises analyzing the total protein concentration of the first urine sample by Bradford protein assay, enzymatic method, bicinchoninic acid assay, Biuret method, Lowry method, UV absorbance method, or Pyrogallol red method.

According to some embodiments of the present disclosure, the step of measuring the creatinine concentration and the albumin concentration of the second urine sample comprises substeps of mixing the second urine sample and a diluent in a mixing reservoir and then distributing into a first diluted urine sample and a second diluted urine sample; measuring a creatinine concentration of the first diluted urine sample; and measuring an albumin concentration of the second diluted urine sample.

According to some embodiments of the present disclosure, the substeps of mixing the second urine sample and the diluent in the mixing reservoir and then distributing into the first diluted urine sample and the second diluted urine sample comprises mixing the second urine sample and the diluent in the mixing reservoir to form a diluted urine sample; and distributing the diluted urine sample into the first diluted urine sample and the second diluted urine sample.

According to some embodiments of the present disclosure, the substep of measuring the creatinine concentration of the first diluted urine sample comprises analyzing the creatinine concentration of the first diluted urine sample by Jaffe reaction or enzymatic method.

According to some embodiments of the present disclosure, the substep of measuring the albumin concentration of the second diluted urine sample comprises analyzing the albumin concentration of the second diluted urine sample by turbidimetric inhibition immunoassay, salicylic acid method, trichloroacetic acid method, dye-binding method, Bromcresol Green method, or benzethonium chloride method.

Another aspect of the present disclosure is to provide a system having a sample reservoir, a diluent reservoir, a mixing reservoir, a total protein detection reservoir, a creatinine detection reservoir, an albumin detection reservoir, a detection device, and a processor. The sample reservoir is configured to receive a urine sample having a first urine sample and a second urine sample. The diluent reservoir is configured to store a diluent. The mixing reservoir is configured for receiving and mixing the diluent and the second urine sample to form a first diluted urine sample and a second diluted urine sample. The creatinine detection reservoir is connected to the mixing reservoir and is for receiving the first diluted urine sample. The albumin detection reservoir is connected to the mixing reservoir and is for receiving the second diluted urine sample. The mixing reservoir is in communication with the sample reservoir, the diluent reservoir, the creatinine detection reservoir, and the albumin detection reservoir. The mixing reservoir is configured to mix the second urine sample from the sample reservoir with the diluent and then introduce a mixture of the second urine sample and the diluent to the creatinine detection reservoir and the albumin detection reservoir. The total protein detection reservoir is connected to the sample reservoir and is for receiving the first urine sample. The detection device is configured to measure a total protein concentration of the first urine sample, a creatinine concentration of the first diluted urine sample, and an albumin concentration of the second diluted urine sample. The processor is configured to calculate a urine protein to creatinine ratio (UPCR) and a urine albumin to creatinine ratio (UACR), in which the UPCR is defined as the ratio of the total protein concentration to the creatinine concentration, while the UACR is defined as the ratio of the albumin concentration to the creatinine concentration.

According to some embodiments of the present disclosure, the first urine sample comprises about 33% to about 80% of the total volume of the urine sample, the first diluted urine sample comprises about 13.3% to about 44.7% of the total volume of the urine sample, and the second diluted urine sample comprises about 6.7% to about 22.3% of the total volume of the urine sample.

### BRIEF DESCRIPTION OF THE DRAWINGS

The present disclosure is best understood from the following detailed description when read with the accompanying figures. It is emphasized that, in accordance with the standard practice in the industry, various features are not drawn to scale and are used for illustration purposes only. In fact, the dimensions of the various features may be arbitrarily increased or reduced for clarity of discussion.

FIG. 1 depicts a flow chart of a determination method according to some embodiments of the present disclosure.

FIG. 2 depicts a schematic view of a determination system according to some embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting.

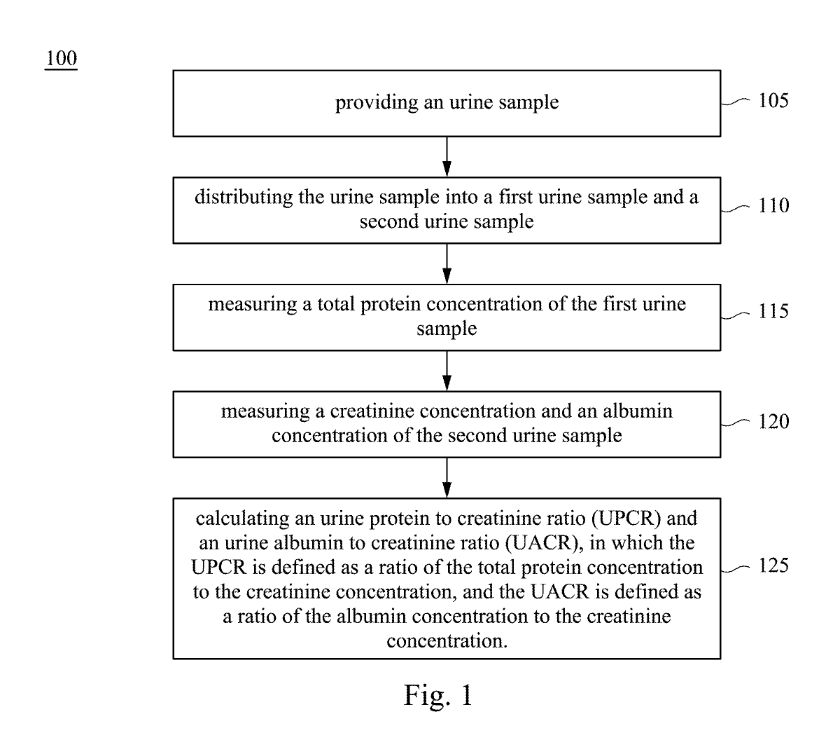

FIG. 1 depicts a flow chart of a determination method 100 according to some embodiments of the present disclosure. The determination method 100 may include steps of: providing or receiving a urine sample (step 105); distributing the urine sample into a first urine sample and a second urine sample (step 110); measuring a total protein concentration of the first urine sample (step 115); measuring a creatinine concentration and an albumin concentration of the second urine sample (step 120); and calculating a urine protein to creatinine ratio (UPCR) and a urine albumin to creatinine ratio (UACR), in which the UPCR is defined as the ratio of the total protein concentration to the creatinine concentration, and the UACR is defined as the ratio of the albumin concentration to the creatinine concentration.

At step 105, a urine sample is provided. The urine sample as described herein may refer to urine excretion of an individual. In some embodiments, the individual includes human and other animals, that is, the urine sample is not limited to be human urine. In some embodiments, the urine sample is 24-hour urine, which refers to the urine of an individual collected over a full 24-hour period. For example, the first time urine of an individual after getting up is discarded, and thereafter the individual's urine is collected over a full 24-hour period. 24-hour urine may reflect the overall pattern of the urine sample in 24 hours and plays a role to increase the detection accuracy.

At step 110, the urine sample is distributed into a first urine sample and a second urine sample. The urine sample may be divided into a plurality of portions based on the analytical items of interest. In some embodiments, based on the total volume of the urine sample, the step of distributing the urine sample into the first urine sample and the second urine sample includes distributing about 33% to about 80% of the urine sample by volume as the first urine sample, and distributing about 20% to about 67% of the urine sample by volume as the second urine sample. For some particular analyte, if the specimen (for example, a urine sample) is analyzed under the original concentration, some impurities or components in the specimen may interfere with the test results. Therefore, the specimen has to be diluted before test. In other words, for those particular analytes, less amount of the specimen is required at the beginning if the specimen is to be diluted. It is noted that although the determination method of the present embodiment has excellent sensitivity, the volume ratio of each portion is adjustable to achieve better test results. For example, in some embodiments, the first urine sample is for analyzing the total protein in the urine sample, while the second urine sample is for analyzing the creatinine and the albumin in the urine sample. Therefore, a volume percentage of the first urine sample may be greater than that of the second urine sample, based on the total volume of the urine sample. Accordingly, in some embodiments, the step of distributing the urine sample into the first urine sample and the second urine sample includes distributing about 60%, 70%, or 80% of the urine sample by volume as the first urine sample, and distributing about 20%, 30%, or 40% of the urine sample by volume as the second urine sample.

The portions from the urine sample may be subjected to different determination methods. At step 115, the total protein concentration of the first urine sample is measured. In some embodiments, the total protein concentration of the first urine sample is measured by means of Bradford protein assay, enzymatic method, bicinchoninic acid assay, Biuret method, Lowry method, UV absorbance method, or Pyrogallol red method.

At step 120, a creatinine concentration and an albumin concentration of the second urine sample are measured. In some embodiments, the creatinine concentration of the second urine sample is measured by using Jaffe reaction or enzymatic method. In some embodiments, the albumin concentration of the second urine sample is measured by using turbidimetric inhibition immunoassay, salicylic acid method, trichloroacetic acid method, dye-binding method, Bromcresol Green method, or benzethonium chloride method.

In addition, as described above, in some cases, the second urine sample may be diluted before test. In some embodiments, the measuring of the creatinine concentration and the albumin concentration of the second urine sample includes mixing the second urine sample and a diluent in a mixing reservoir; and then distributing the mixture of the second urine sample and the diluent into a first diluted urine sample and a second diluted urine sample. For example, the second urine sample and the diluent are mixed in the mixing reservoir to form a diluted urine sample. Next, the diluted urine sample is distributed into the first diluted urine sample and the second diluted urine sample. In other words, the diluted urine sample is distributed into two parts, i.e., the first diluted urine sample and the second diluted urine sample.

Finally, a creatinine concentration of the first diluted urine sample and an albumin concentration of the second diluted urine sample are measured. In some embodiments, the creatinine concentration of the first diluted urine sample is measured by the same method of measuring the creatinine concentration of the second urine sample, while the albumin concentration of the second diluted urine sample is measured by the same method of measuring the albumin concentration of the second urine sample.

The total protein concentration, the creatinine concentration and albumin concentration are further processed. At step 125, a urine protein to creatinine ratio (UPCR) and a urine albumin to creatinine ratio (UACR) are calculated, in which the UPCR is defined as the ratio of the total protein concentration to the creatinine concentration, and the UACR is defined as the ratio of the albumin concentration to the creatinine concentration. Accordingly, in the above-mentioned embodiment, after the urine sample is collected, the total protein concentration, the creatinine concentration, and the albumin concentration of the urine sample can be rapidly measured using the same determination procedure and method, so that the UPCR and the UACR are obtained. The UPCR and UACR may help to diagnose CKD and determine CKD stages. If the UACR is above the normal level, the individual is likely to experience albuminuria, which is common at stage 2. If the UPCR is above the normal level, the individual is likely to experience proteinuria, which is common at stage 3. The advantages of obtaining the UACR and UPCR in the same determination method can provide more robust proof for CKD diagnosis.

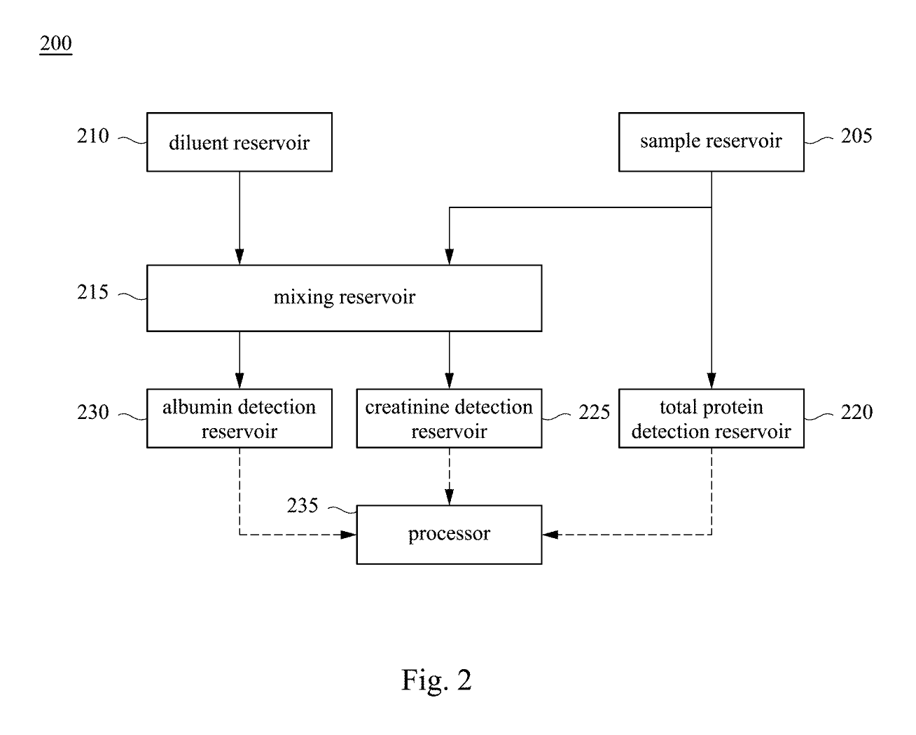

Reference is made to FIG. 2. FIG. 2 depicts a schematic view of a determination system 200 according to some embodiments of the present disclosure. The determination system 200 has a sample reservoir 205, a diluent reservoir 210, a mixing reservoir 215, a total protein detection reservoir 220, a creatinine detection reservoir 225, an albumin detection reservoir 230, a detection device (not shown), and a processor 235. The sample reservoir 205 is configured to receive a urine sample. A portion of the urine sample in the sample reservoir 205 is introduced to the total protein detection reservoir 220 and is referred to as “first urine sample” hereinafter. In addition, the remaining portion of the urine sample is introduced to the mixing reservoir 215 from the sample reservoir 205 and is referred to as “second urine sample.” In some embodiments, details of the urine sample are described above and therefore are not repeated herein. In some embodiments, the volume ratio of the first urine sample and the second urine sample is adjustable. For example, the first urine sample may comprise about 33% to about 80% of the total volume of the urine sample, and the second urine sample may comprise about 20% to about 67% of the total volume of the urine sample.

The diluent reservoir 210 is configured to store a diluent. In some embodiments, the diluent may help to adjust the concentration of the urine sample, solubility of a particular component and other properties that may enhance the accuracy and operability of the test.

The mixing reservoir 215 is configured to receive and mix the diluent and the second urine sample to form a diluted urine sample. Specifically, the diluent is introduced to the mixing reservoir 215 from the diluent reservoir 210 and mixed with the second urine sample in the mixing reservoir 215 to form the diluted urine sample. A portion of the diluted urine sample is introduced to the creatinine detection reservoir 225 and is referred to as “first diluted urine sample.” The remaining portion of the diluted urine sample is introduced to the albumin detection reservoir 230 and is referred to as “second diluted urine sample.”

The creatinine detection reservoir 225 and the albumin detection reservoir 230 are connected to the mixing reservoir 215. The creatinine detection reservoir 225 is configured to receive the first diluted urine sample, while the albumin detection reservoir 230 is configured to receive the second diluted urine sample. In other words, in the determination system 200, the urine sample is divided into three specimens, i.e., the first urine sample (which is introduced to the total protein detection reservoir 220), the first diluted urine sample (which is introduced to the creatinine detection reservoir 225), and the second diluted urine sample (which is introduced to the albumin detection reservoir 230). In some embodiments, the first urine sample comprises about 33% to about 80% of the total volume of the urine sample, the first diluted urine sample comprises about 13.3% to about 44.7% of the total volume of the urine sample, and the second diluted urine sample comprises about 6.7% to about 22.3% of the total volume of the urine sample. In summary, the mixing reservoir 215 is in communication with the sample reservoir 205, the diluent reservoir 210, the creatinine detection reservoir 225, and the albumin detection reservoir 230.

As described above, the total protein detection reservoir 220 is connected to the sample reservoir 205 so as to receive the first urine sample. It is noted that the total protein detection reservoir 220 is not connected with the mixing reservoir 215, such that the first urine sample directed to the total protein detection reservoir 220 from the sample reservoir 205 is not diluted.

The determination system 200 may further include one or more concentration detection device (not shown) for measuring the total protein concentration of the first urine sample, the creatinine concentration of the first diluted urine sample, and the albumin concentration of the second diluted urine sample. The detection device may be disposed in the determination system 200, such that the total protein concentration, the creatinine concentration, and the albumin concentration are measured when the first urine sample, the first diluted urine sample, and the second diluted urine sample is introduced to the total protein detection reservoir 220, the creatinine detection reservoir 225, and the albumin detection reservoir 230 respectively.

The processor 235 is configured to process the total protein concentration, the creatinine concentration, and the albumin concentration to calculate a urine protein to creatinine ratio (UPCR) and a urine albumin to creatinine ratio (UACR), in which the UPCR is defined as the ratio of the total protein concentration to the creatinine concentration, while the UACR is defined as the ratio of the albumin concentration to the creatinine concentration. For example, when the total protein concentration, the creatinine concentration, and the albumin concentration are measured by the detection device, the detection results are transmitted to the processor 235. Therefore, the UACR and UPCR can be calculated by the determination system 200, which is beneficial to the prevention and treatment of early-stage CKD. In addition, it is noted that the determination method and the determination system of the present embodiment facilitate the operation of measuring UACR and UPCR. For example, the determination system 200 can be applied to a miniature measuring plate, and therefore it may serve as a small and portable device which is suitable to be used in rapid clinical diagnosis.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A determination method, comprising steps of:
providing or receiving a urine sample;
distributing the urine sample into a first urine sample and a second urine sample;
measuring a total protein concentration of the first urine sample;
measuring a creatinine concentration and an albumin concentration of the second urine sample; and
calculating a urine protein to creatinine ratio (UPCR) and a urine albumin to creatinine ratio (UACR), wherein the UPCR is defined as a ratio of the total protein concentration to the creatinine concentration, and the UACR is defined as a ratio of the albumin concentration to the creatinine concentration.

2. The method of claim 1, wherein the step of distributing the urine sample into the first urine sample and the second urine sample comprises:
distributing 33% to 80% of the urine sample by volume as the first urine sample; and
distributing 20% to 67% of the urine sample by volume as the second urine sample.

3. The method of claim 1, wherein the step of measuring the total protein concentration of the first urine sample comprises analyzing the total protein concentration of the first urine sample by Bradford protein assay, enzymatic method, bicinchoninic acid assay, Biuret method, Lowry method, UV absorbance method, or Pyrogallol red method.

4. The method of claim 1, wherein the step of measuring the creatinine concentration and the albumin concentration of the second urine sample comprises substeps of:
mixing the second urine sample and a diluent in a mixing reservoir and then distributing into a first diluted urine sample and a second diluted urine sample;
measuring a creatinine concentration of the first diluted urine sample; and
measuring an albumin concentration of the second diluted urine sample.

5. The method of claim 4, wherein the substep of mixing the second urine sample and the diluent in the mixing reservoir and then distributing into the first diluted urine sample and the second diluted urine sample comprises:
mixing the second urine sample and the diluent in the mixing reservoir to form a diluted urine sample; and
distributing the diluted urine sample into the first diluted urine sample and the second diluted urine sample.

6. The method of claim 4, wherein the substep of measuring the creatinine concentration of the first diluted urine sample comprises analyzing the creatinine concentration of the first diluted urine sample by Jaffe reaction or enzymatic method.

7. The method of claim 4, wherein the substep of measuring the albumin concentration of the second diluted urine sample comprises analyzing the albumin concentration of the second diluted urine sample by turbidimetric inhibition immunoassay, salicylic acid method, trichloroacetic acid method, dye-binding method, Bromcresol Green method, or benzethonium chloride method.

8. A determination system, comprising:
a sample reservoir configured to receive a urine sample, wherein the urine sample comprises a first urine sample and a second urine sample;
a diluent reservoir configured to store a diluent;
a mixing reservoir configured for receiving and mixing the diluent and the second urine sample to form a first diluted urine sample and a second diluted urine sample;
a creatinine detection reservoir connected to the mixing reservoir, wherein the creatinine detection reservoir is for receiving the first diluted urine sample;
an albumin detection reservoir connected to the mixing reservoir, and is for receiving the second diluted urine sample, wherein the mixing reservoir is connected with the sample reservoir, the diluent reservoir, the creatinine detection reservoir, and the albumin detection reservoir, and the mixing reservoir is configured to mix the second urine sample from the sample reservoir with the diluent and then introduce a mixture of the second urine sample and the diluent to the creatinine detection reservoir and the albumin detection reservoir;
a total protein detection reservoir connected to the sample reservoir, wherein the total protein detection reservoir is for receiving the first urine sample;
a detection device configured to measure a total protein concentration of the first urine sample, a creatinine concentration of the first diluted urine sample and an albumin concentration of the second diluted urine sample; and
a processor configured to process the total protein concentration, the creatinine concentration, and the albumin concentration to calculate a urine protein to creatinine ratio (UPCR) and a urine albumin to creatinine ratio (UACR), wherein the UPCR is defined as the ratio of the total protein concentration to the creatinine concentration, while the UACR is defined as the ratio of the albumin concentration to the creatinine concentration.

9. The determination system of claim 8, wherein the first urine sample comprises 33% to 80% of a total volume of the urine sample, the first diluted urine sample comprises 13.3% to 44.7% of the total volume of the urine sample, and the second diluted urine sample comprises 6.7% to 22.3% of the total volume of the urine sample.
